SEQUENCE LISTING (7 sequences)

SEQ ID NO: 1 - DNA, 3374 bp, Homo sapiens
gccctttcttatgagcatgcctgtgttgggttgacagtgagggtaataatgacttgttggttgattgtagatatagggctctcccttgcaaggtaattaggctccttaaattacctgtaagattttcttgccacagcatccattctggttaggctggtgatcttctgagtagtgatagattggttggtggtgaggtttacaggtgttcccttctcttactcctggtgttggctacaatcaggtggcgtctagagcagcatgggacaggtgggtaaggggagtcttctcattatgcagaagtgatcaacttaaatctctgtcagatctacctttatgtagcccggcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggggactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcccctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcctttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaa

SEQ ID NO: 2 - DNA, 3388 bp, Mus musculus
gtcgtagctatccctggaatgaggcgcttacacattttatttctttcatgcctgacataaagtctggcccttgctcgctcctgccccccgtccaaatggctcggcccgcggaacgccccatcttccaggcacattgagagccggagtcttggaggagtttagggtggtgattctacaacggcgactagcaagtggcgggcttcagccctttcccgctgctctcctggtcgcgaccacacgtcacagctctcgctcgttccggttgctcgcgcacgggccccagaagcgcaggcgagatcggagcgcgcaaagagaacttggtacggtccactccgccgcgccccgcgccgccggaagtgaggtgtcttacccccgaagttccggttcgcagggggtggggagtgttgttaaccggagcggctgccgcagtcgcggtgattgagcgtgctcgcggcgctgggctcctggtctctgggccagggcgatgttccgcaccgcagtgatgatggcggccagcctggcgctaaccggggcagtggtggctcatgcctactacctcaaacaccagttctaccccactgtagtgtatttgaccaagtccagccccagcatggcagtcctgtacatccaggcctttgtccttgtcttcctcttgggcaaggtgatgggcaaggtgttcttcgggcagctgagggcagcagagatggagcaccttctggaacggtcctggtacgctgttactgagacttgtttggccttcaccgtttttcgggatgacttcagccctcgctttgtggcgctctttacgctgctcctcttcctcaaatgtttccattggttggctgaagaccgtgtggactttatggaacgcagccccaacatctcctggctcttccactgccgcatcgtctctctcatgtttctcctgggtatcctggacttcctcttcgtcagccacgcttatcacagcatcctgacccgtggggcttctgtgcagctggtatttggctttgagtacgccattctgatgaccatggtgcttaccatcttcatcaagtatgtgctgcactccgtggacctccagagcgagaacccctgggacaacaaggctgtatacatgctctacacggagctgtttacaggcttcatcaaggtcctgctgtacatggccttcatgaccatcatgatcaaggtgcacacattcccactctttgccattaggcccatgtacctggccatgaggcagttcaagaaagctgtgacagatgccatcatgtctcgccgagccatccgcaacatgaacacactgtacccagatgccacccccgaggagctccaggcagtggataatgtctgtatcatctgcagagaagaaatggtgactggtgctaagagattgccttgcaaccacatctttcacacgagctgcctgcgctcctggttccagagacagcagacctgcccgacatgccgcatggatgtcctgcgggcatcgttgccagcccagtcaccaccacctcctgagcctgctgaccaaggaccaccccccgcccctcatccccaaccgctgctgccacagccccctaatttcccccagggcctcctgcctccttttcctccaggcatgttcccactgtggcccccaatgggtccctttccacctgtcccgcctcccccaagctcaggagaggctgcggcccctccacccaccagtacagccgtttctcggcctagtggagcagccaccaccacagctgctggcaccagtacttctgccccagcacctgggtctgtacctggcccagaggctggtcctgcccccggcttccctttccctcctccttggatgggtatgcctctgcctccaccttttgccttccccccaatgcctgtgccccctgcgggctttgctggcctaaccccagaggagctgcgagcactagagggccatgagcggcagcacctggaggcccggctgcagagtctgcgcaacatacacacactactggatgctgccatgcttcaaatcaaccagtacctcactgtgctggcttccctggggccccccaggccagctacttcagtgaaccccactgaagagactgcctctacagtggtatctgctgccccttccaccagcgcccccagctctgaggctcctaccccgtctccgggagcttccccaccaattcctgaagcagaaaagcctcctgctcctgagtcagtgggcattgtagaggagcttcccgaggacggagagcctgatgctgcagaactccgccggcgtcgcctgcagaagctggagtcccctgttgcccactgacactgcccagacctggccctgttctcttgagtggccctcactggaacacgtcctgccatcaagtgccagctccctctctgcttgcaccagggagtaatagccccagttgagaaagacttggcaggatctctgaggatcaaggagaagtgtctgggcttccagttgatccatccccagtgcccctggcagccatggagatactggtcagctctaacctccctccacttctgccatgttcaactggggccttcaaagtagaagctgaatctctggtaagccttctcttccatgctttctgggagaaggtgaagcccctccaagccctgcttgtgagtatgggaccatgctgcagtgccgaacagtattagcttctgttcccaagtgtggaaacccagaggggctgaagacagaccaggaccttgccccaccctcctgccaagactggtaccagtctctttcctctagcccagtcttcccagaacccctttgtgatggtggctgtgccccccgaagccctgtggcatttccatgtcttactggcaaccacacaactcagggaaaggagtgcctgggggtggggcacaggcgggcagcactgagggaccctgccctgcccctccccagctctttccccatctcacccagcagccactgcctggtgggcctggctaagggtgtgtgctgctccttaaaccactgctccccagaacccaaggcaggccacctccaacctgtgggatgtcgtcaggattggaactattctgtacctactggctttgggcttaaattttgtcttctgaatttgaatgcttgaccccaggaaggaggagcaggtgtggggctaggtacctggacttcgcagtttagaacaagctctgggccgggccgggccaggccaggcctagggagccaaggcctagctgctgcttccttcttttggttttgtgttacaggagtttctggagagtttcagatgattatttaatttgtaaatattgtataaattttaatagcttaaattgtatatacagctcaataaaaacttgcattaaaaaaaaaaaaaaa

SEQ ID NO: 3 - DNA, 19 bp, Homo sapiens
cgttcctggtacgccgtca

SEQ ID NO: 4 - DNA, 19 bp, Mus musculus
gaaatggtgactggtgcta

SEQ ID NO: 5 - DNA, 19 bp, Artificial sequence
  synthetic DNA
ggctacgtccaggagcgca

SEQ ID NO: 6 - DNA, 20 bp, Artificial
  synthetic DNA
gcgccgccggaagtgaggtg

SEQ ID NO: 7 - DNA, 20 bp, Artificial
  synthetic DNA
cacctcacttccggcggcgc

(19) United States
(12) Patent Application Publication
(10) Pub. No.: US 2007/0219150 A1
(43) Pub. Date: Sep. 20, 2007

(54) Nerve Cell Differentiation Inducer

(75) Inventors: Toshihiro Nakajima, Yokohama-shi (JP); Satoshi Yamasaki, Yokohama-shi (JP); Naoko Yagishita, Yokohama-shi (JP); Daijuro Tonaki, Yokohama-shi (JP); Yukihiro Kato, Yamato-shi (JP)

Correspondence Address: DICKSTEIN SHAPIRO LLP, 1177 AVENUE OF THE AMERICAS (6TH AVENUE), NEW YORK, NY, 10036-2714, US

(73) Assignee: Locomogene, Inc., Kanagawa (JP)

(21) Appl. No.: 10/588,290

(22) PCT Filed: Feb. 4, 2005
(86) PCT No.: PCT/JP05/02106

§ 371 Date: Feb. 20, 2007

(30) Foreign Application Priority Data
Feb. 6, 2004 (JP) 2004-031320

(51) Int. Cl. A61K 31/7052; C12N 5/00

(52) U.S. Cl. 514/44; 435/377

(57) ABSTRACT

The present invention provides a nerve cell differentiation inducing drug containing a Synoviolin expression inhibitor which is a useful drug for treatments of neural disorders, particularly Alzheimer's disease, Parkinson's disease, peripheral nerve disorders or spinal injury. As Synoviolin expression inhibitors, there are siRNA against gene coding Synoviolin (SEQ ID No. 1 or 2), or shRNA, a decoy nucleic acid that inhibits the promoter activity by binding to the transcription factor of the promoter of the Synoviolin gene or antisense oligonucleotide against gene coding the Synoviolin. The nerve cell differentiation inducing drug of the present invention is used for the treatment of neural disorders including Alzheimer's disease, Parkinson's disease, peripheral nerve disorders or spinal injury.

### TECHNICAL FIELD

This invention relates to a nerve cell differentiation inducing drug containing a Synoviolin expression inhibitor.

### BACKGROUND ART

Synoviolin is a protein discovered as a membrane protein located in synovial cells from rheumatoid arthritis patients (WO02/05207). Based on the studies using transgenic animals, it became clear that this factor is directly related to generation of bones and joints and onsets of arthropathy. Synoviolin is considered to be the protein contributing to normal osteogenesis and development of the extremities.

Synoviolin is an ubiquitin ligase involved in endoplasmic reticulum associated degradation (ERAD). Recently, its has been clarified that causative genes of the neural degenerative diseases such as familial Alzheimer's disease and Parkinson's disease are associated to ERAD(Nakagawa T, Zhu H, Morishima N, Li E, Xu J, Yankner B A, Yuan J., Caspase-12 mediates endoplasmic-reticulum-specific apoptosis and cytotoxicity by amyloid-beta, Nature, Jan. 6, 2000: 403(6765): 98-103).

Alzheimer's disease is one of the diseases that have attracted high interest in our aging society. The most important characteristics are observation of aging spots in the brain, namely fibrous β-amyloid protein (Aβ).

However, association of Synoviolin to neural degenerative diseases has not yet been clearly understood.

### DISCLOSURE OF THE INVENTION

Purpose of the present invention is to provide drugs that are useful for treatments of neural disorders, particularly Alzheimer's disease or Parkinson's disease, peripheral nerve disorders and spinal injury. The inventors earnestly investigated the aforementioned problems and discovered that when expression of Synoviolin is inhibited, nerve cell differentiation of primitive cells is induced. This finding led us to achieve the present invention.

That is, the present invention is as follows.

(1) A nerve cell differentiation inducing drug containing a Synoviolin expression inhibitor.

As inhibitors for expression of Synoviolin, for example siRNA or shRNA against genes coding Synoviolin (SEQ ID No. 1 or 2), decoy nucleic acids inhibiting Synoviolin gene promoter activity or antisense oligonucleotides against genes coding Synoviolin (SEQ ID NO. 1 and 2) are available. siRNA can target a portion of the sequence among the base sequences indicated by SEQ ID No. 1 (e.g., sequence indicated by SEQ ID No. 3), or a portion of the sequence among the base sequences indicated by SEQ ID No. 2 (e.g., sequence indicated by SEQ ID No. 4).

Decoy nucleic acids include the following decoy nucleic acids as shown in (a) or (b).

(a) Decoy nucleic acids consisting of a base sequence indicated by SEQ ID No. 6 or No. 7.

(b) Decoy nucleic acids consisting of a base sequence after deleting 1 or several bases, substituting or inserting base sequences in the base sequence indicated by SEQ ID No. 6 or No. 7 and having a function of inhibiting Synoviolin gene promoter activity.

Decoy nucleic acids can be those shown by the following (a) or (b)

(a) Decoy nucleic acids consisting of a base sequence indicated by SEQ ID No. 6 and No. 7.

(b) Decoy nucleic acids consisting of a base sequence after deleting 1 or several bases, substituting or inserting base sequences in the base sequence indicated by SEQ ID No. 6 and No. 7 and having a function of inhibiting Synoviolin gene promoter activity.

The aforementioned decoy nucleic acids inhibit Synoviolin expression by inhibiting Synoviolin gene promoter activity.

Anti-sense oligonucleotides can target a portion of base sequence among the base sequences identified by SEQ ID No. 1 and 2.

The nerve cell differentiation drug of the present invention is used for the treatment of Alzheimer's disease, Parkinson's disease, peripheral nerve disorders and spinal injury.

(2) A method of inducing nerve cell differentiation which is characterized by inhibiting expression of Synoviolin.

### BRIEF DESCRIPTION OF THE DRAWINGS

FIG. 1 is a diagram showing that nerve cell differentiation is induced by inhibiting the expression of Synoviolin by siRNA.

### BEST MODE FOR CARRYING OUT THE INVENTION

This invention is explained precisely below.

The purpose of the present invention is to clarify the association of Synoviolin to the neural degenerative diseases. Initially, we performed functional analysis of Synoviolin in the nerve cells. As a result, when the expression of Synoviolin is inhibited, there is cellular differentiation in the nerve cells.

<Differentiation of Nerve Cells>

Nerve cell differentiation means either a certain type of cells undergo morphological alternations to the nerve cells, and that cells maintain differentiation until functions as nerve cells can be achieved. When a certain types of cells undergo morphological changes in the nerve cells or functional changes, it is determined that the differentiation of nerve cells is induced. However, the origins or kinds of cells are not limited, which differentiated into nerve cells. They can be human derived cells (human patients derived cells or cells derived from healthy adults), or cells derived from rats or mice. In addition, kinds of cells can be undifferentiated nerve cells, stem cells and established cells. The stem cells can be used for the treatment of neural diseases by implanting by ex vivo differentiation of the nerve cells (regenerative healthcare). Examples of established cells include rat PC-12 cells, mouse Neuro2a and human NB-1, etc.

<Inhibition of Expression of Synoviolin>

The way of inhibiting the expression of Synoviolin is not particularly limited. For example, RNA interference (RNAi), decoy nucleic acids or antisense oligonucleotides can be utilized.

1. RNA Interference

siRNA (small interfering RNA) is designed and synthesized for the Synoviolin gene to introduce it into the cell to induce RNAi. RNAi is dsRNA (double-strand RNA) which is specific and selective towards the target gene, and also by cutting the target gene the expression can be inhibited more efficiently. For example, when dsRNA is transfected, the expression of genes contain homo sequence to the dsRNA is suppressed (knock down). siRNA can be designed as described below.

(a) In the case of genes coding Synoviolin, they are not particularly restricted.

All arbitrary areas can be selected as candidates. For example, in the case of humans, an arbitrary area of GenBank Accession number AB024690 (SEQ ID No. 1) can be selected as a candidate. In the case of mice, an arbitrary area of Accession number NM_{—}028769 (SEQ ID No. 2) can be selected as candidate.

(b) From the selected area, a sequence starting with AA is selected and the length of the sequence ranges from 19 to 25 bases, and preferably from 19 to 21 bases. The GC content in the sequence is selected to be from 40 to 70%. Specifically, among the base sequences indicated by SEQ ID No. 1 and 2, DNA containing the following base sequences can be used as target base sequences of siRNA.

Synoviolin siRNA1:

(SEQ ID No.3)

CGT TCC TGG TAC GCC GTC A
Synoviolin siRNA2:

(SEQ ID No.4)

GAA ATG GTG ACT GGT GCT A

Synoviolin siRNAI: Among the base sequences indicated in SEQ ID No. 1, the 640 to 658^{th} areas are targets. Synoviolin siRNA2 is a base sequence aiming at the 1373 to ₁₃₉₁^{st} areas of the base sequence shown for the SEQ ID No. 2.

As a method for tranfecting siRNA into the cells, siRNA synthesized in vitro is linked to plasmide DNA or a method of annealing two-stranded RNA is available.

In order to achieve the RNAi effect, shRNA can be used. shRNA is called a short hair pin RNA (short hair pin RNA). To form a complementary chain between a portion of the area of a single strand and the other area, it is an RNA molecule having a stem loop structure.

shRNA can be designed to form a stem loop structure as its part. For example, if a sequence of a certain area is sequence A and the complementary chain to the sequence A is sequence B, sequence A+spacer+sequence B in this order form a single RNA chain and a total of 45 to 60 bases is designed. Sequence A is a sequence of a part of the area in the target Synoviolin gene (SEQ ID No. 1 and 2, but the target area is not particularly limited and an arbitrary area can be selected. The length of the sequence A is 19 to 25 bases and preferably 19 to 21 bases.

2. Decoy Nucleic Acids

A decoy nucleic acid in the present invention implies “a decoy nucleic acid” including the binding site for a transcription factor. If this nucleic acid is transfected into the cell, the transcription factor binds to this nucleic acid so that the binding of the transcription factor to the original genome binding site is competitively inhibited. As a result, expression of the transcription factor is inhibited. Typically, a decoy nucleic acid is a nucleic acid and its analogs and contains at least one nucleic acid to be bonded to the transcription factor.

Desirable examples of decoy nucleic acid are as follows. For example, a nucleic acid capable of binding to a transcription factor that binds to EBS which is a Ets binding site carrying out constitutive expression in the Synoviolin promoter; a nucleic acid capable of binding to a transcription factor that binds to the ABS (AML binding side) which is a promoter's transcription factor binding site; a nucleic acid capable of binding to a transcription factor that binds to the SBS (Sp1 binding site) which is a promoter's transcription factor binding site; oligonucleotides containing their complementary base pairs, their mutants or nucleic acids containing them in the molecule. Decoy nucleic acids can be designed by forming a single strand based on the above-mentioned sequences EBS, ABS or SBS, or double strands comprising of its complementary strand. The length is not particularly restricted, but a desirable length ranges from 15 to 60 bases and preferably from 20 to 30 bases.

In the present invention a nucleic acid capable of binding to a transcription factor binding to EBS (SEQ ID No. 6) and/or its complementary strand (SEQ ID No. 7) can be used preferably as a decoy nucleic acid.

Nucleic acids can be DNA or RNA, or the nucleic acid can contain modified nucleic acids or a pseudo nucleic acid

The nucleic acids containing nucleic acid, mutant, or nucleic acid containing these within the molecule can be made of a single strand or double strands. In addition, it can be cyclic or linear. A mutant is made of a base sequence from which one to several bases in the aforementioned decoy nucleic acid (For example, 1 to 10, 1 to 5, or I to 2) are deleted, or substituted, or one to several bases in the aforementioned decoy nucleic acid (For example, 1 to 10, 1 to 5, or 1 to 2) are inserted, which has a function of inhibiting the promoter activity of the Synoviolin gene, namely it is called a nucleic acid having a function of binding with a transcription factor. It can be a nucleic acid containing one or more of the aforementioned base sequences.

The decoy nucleic acid used in the present invention can be manufactured by a chemical synthesis or a biochemical synthesis that is known to those in the art. For example, a nucleic acid synthesis method using a common DNA synthesis apparatus can be employed as a gene recombinant technology. In addition, after separating or synthesizing a template sequences, a PCR method or a gene amplification method by using cloning vector can be used. Moreover, according to these methods, the nucleic acid obtained can be cut with a limiting enzyme, or a desired nucleic acid can be manufactured by binding a DNA ligase. Moreover, in order to achieve a stable decoy nucleic acid within the cell, chemical modifications such as alkylation and acylation can be added to the base sequence. A method of preparing a mutant from the decoy nucleic acid, a known method that is known to those in the art can be employed. For example, it can be synthesized by a site-specific mutation induction method. A site-specific mutation induction method is a known to those in the art and a commercial kit can be purchased.

For example, the following commercial kits can be used: GeneTailor™ Site-Directed Mutagenesis System (Invitrogen Corp.), TaKaRa Site-Directed Mutagenesis System (Mutan-K, Mutan-Super Express Km, etc. (Takara Bio Corp)).

For analysis of the transcription activity for the promoter when using a decoy nucleic acid, the following generally known method can be employed: e.g., Luciferase assay, Gel Mobility Shift assay, and CAT assay. A kit is also available to carry out these assay methods. (e.g., Promega dual luciferase assay kit.)

For example, in the case of the Luciferase assay, a firefly luciferase gene is connected as a reporter upstream to the start point of transcription of the target gene. In order to correct intercellular introduction efficacy for the assay, cytomegalo virus (CMV) β-galactosidase (β-gal) gene as a reporter can be introduced to the cell along with the vector connected downstream to the promoter at the same time.

For the transfection to the cell, for example, calcium phosphate method can be used. If a vector is transfected to the cells, the cells can be cultured for a specified time and then recovered. Subsequently, the cells are lysed by freeze and thaw, luciferase and β-galactosidase activity was measured using a fixed amount of cell extract.

3. Antisense Oligonucleotides

An antisense oligonucleotide in this invention is a nucleotide having a complementary sequence relative to the base sequence coding the Synoviolin. When this antisense oligonucleotide forms a hybrid with all or a portion of the base sequence coding Synoviolin gene, expression of the Synoviolin gene is inhibited so that synthesis of Synoviolin protein can be inhibited efficiently. If this invention's antisense oligonucleotide hybridizes with this invention's RNA of the Synoviolin gene or the entire or a portion of RNA in association with this invention's Synoviolin gene, the aforementioned RNA synthesis or functions are inhibited or expression of this invention's Synoviolin gene can be adjusted and controlled via interaction with this invention's Synoviolin associated with RNA. A desirable example of an antisense oligonucleotide is presented as complementary sequences to the following sequence: 5′-terminal hair pin loop of this invention's Synoviolin gene; 5′-6-base pair repeat, 5′-terminal non translation area, polypeptide translation start, protein code area, ORF translation stop codon, 3′-nontranslation area, 3′-Palindrom area, and 3′-hair pin loop. This can be selected as a target area in any area within Synoviolin genes. Specifically, in the case of (a) antisense oligonucleotide directed to inhibition of translation, it is desirable to have a complementary sequence with the base sequence for coding the N-terminal of Synoviolin of this invention (e.g., base sequence near start codon); (b) in the case of antisense oligonucleotide directed to RNA degradation by RNaseH, it is desirable to have a complementary sequence to the base sequence coding Synoviolin gene of this invention including intron. Antisense oligonucleotide can be designed as a single strand, or a double strand including the complementary strand, or as DNA and RNA hybrid based on the aforementioned base sequence. The length is not particularly limited, but it generally ranges from 10 to 40 bases and preferably from 15 to 30 bases.

This invention's antisense oligonucleotide can be DNA or RNA, or the nucleic acid can contain a modified nucleic acid and /or pseudo-nucleic acid. In addition, these nucleic acids, their mutants, or nucleic acids containing these molecules can be single stranded or a double stranded. The mutant means the oligonucleotide sequences that one or several bases (for example, 1 to 10 bases, 1 to 5 bases or 1 to 2 bases) in the aforementioned sequence are deleted or substituted, or one or several bases (for example, 1 to 10 bases, 1 to 5 bases or 1 to 2 bases) in the aforementioned sequence are inserted, which has a function of inhibiting expression of Synoviolin gene. The antisense oligonucleotide itself is hydridized with DNA having a base sequence expressed by SEQ ID No. 1 or No. 2, and the nucleotide containing at least one or more base sequence.

Synthesis, mutation introduction and modification of antisense oligonucleotides used in this invention, can be carried out as described in the section for “decoy nucleic acids”.

The inhibitory activity of antisense oligonucleotide can be investigated using a transformant wherein this invention's antisense oligonucleotide is transfected, and in vivo and in vitro gene expression system using such transformant.

<Pharmaceutical Compositions of the Invention>

shRNA or siRNA, decoy nucleic acids, and antisense oligonucleotides prepared in the present invention are substances that inhibit expression of Synoviolin. Thus, they can be used as pharmaceutical compositions (genetic therapeutic agents for neural disorders) for the purpose of inducting differentiation of nerve cells.

When using the pharmaceutical compositions of the invention as genetic therapeutic agents for neural disorders, indications included diseases in the central nervous systems such as brain (cerebrum, diencephalons, midbrain, and cerebellum) medulla oblongata, spinal cord, and peripheral nervous systems.

Neural disorders include Alzheimer's disease, Parkinson's disease, Huntington's Disease, peripheral nerve disorders and spinal injury.

The aforementioned neural disorders can occur singly or in combination of two or more.

The pharmaceutical compositions of the invention are used in such that shRNA or siRNA, which decoy nucleic acid or antisense oligonucleotides, is incorporated into the infected cells or cells of the target tissue.

The mode of administration of the pharmaceutical compositions of the invention can be either oral or non-oral route. In the case of oral administration, an appropriate drug form can be selected from tablets, pearls, sugar-coated tablets, capsules, liquid agents, gels, syrups, slurries and suspensions. In the case of non-oral administration, via pulmonary administration types (e.g., using a nebulizer, etc.), via nasal administration types, subcutaneous injection types (e.g., ointments, cream agents), and injection types are available. In the case of injection types, agents can be administered systemically or locally, directly or indirectly to the infected areas via various drip fusions such as intravenous injection, intramuscular injection, intraperitoneal injection and subcutaneous injection.

If the pharmaceutical composition of the invention is used as a gene therapy, in addition to direct administration by injection of the composition, a method of administering a vector incorporating a nucleic acid is available. As the aforementioned vectors, adenoviral vector, adeno-associated viral vector, herpes viral vector, vaccinia viral vector, retroviral vector, lentiviral vector, and the like are available. Use of these viral vectors makes administration more efficient. In order to introduce a pharmaceutical composition to a target tissue or organ, it is possible to use a commercial gene transfection kit (e.g., adeno express: Clontech Corp.).

A pharmaceutical composition of the invention can be introduced into a phospholipids endoplasmic reticulum such as a liposome and the liposome can be administered. An endoplasmic reticulum retaining a pharmaceutical composition of the invention is transfected to a specific cell by the lipofection method. The cells obtained are then administered systemically intravenously or intra-arterially. They can be administered locally, for example, to the brain. As lipids to form a liposome structure, phospholipids, cholesterols and nitrolipids can be used. In general, phospholipids are suitable. Natural phospholipids such as phosphatidylcholine, phosphatidylserine, phosphatidylglycerol, phosphatidylinositol, phosphatidylethanolamine, phosphatidic acid, cardiolipin, sphingomyeline, egg yolk lecithin, soybean lecithin, lysolecithin; or their hydrogenated products prepared by an ordinary method are available. In addition, synthetic phospholipids can be used: dicetyl phosphate, distearoylphosphatidylcholine, dipalmitoylphosphatidylserine, eleostearoylphosphatidylcholine, and eleostearoylphosphatidylethanolamine, etc.

A method of producing liposome is not particularly restricted as long as siRNA or shRNA, decoy nucleic acid or antisense oligonucleotides can be retained. A common method includes a reverse phase vaporization method (Szoka, F. et al., Biochim. Biophys. Acta, Vol. 601 559 (1980)), ether injection method (Deamer, D. W.: Ann. N.Y. Acad. Sci., Vol. 308 250 (1978)), surface activation method (Brunner, J. et al.: Biochim. Biophys. Act, Vol. 455 322 (1976)).

Lipids containing these phospholipids can be used singly or two or more kinds can be combined. In this case, if those having an atomic group having positive groups such as ethanolamine and choline in the molecule are used, the degree of association of nucleic acid that is electrically negative can be increased. Besides major phospholipids used when forming these liposomes, additives that are generally known as additives for liposome formation such as cholesterols, stearylamine, and a-tocopherol, etc. can be used. To the liposomes obtained above, membrane fusion promoting substances such as the sendai virus, inactivated sendai virus, membrane fusion promoting proteins purified from sendai virus, and polyethylene glycol, etc. can be used in order to promote intake into the infected cells or the cells in the target tissue.

The pharmaceutical compositions of the invention can be formulated by an ordinary method and can contain medically acceptable carriers. Such carriers can be additives or the following additives are available: water, organic solvents that are medically acceptable, collagen, polyvinyl alcohol, polyvinylpyrrolidone, carboxyvinyl polymer, sodium carboxymethylcellulose, sodium polyacrylate, sodium alginate, water-soluble dextran, sodium carboxymethyl starch, pectin, methylcellulose, ethyl cellulose, xanthan gum, gum Arabic, casein, agar-agar, polyethylene glycol, diglycerin, glycerin, propylene glycol, vaseline, paraffin, stearyl alcohol, stearic acid, human serum albumin, mannitol, sorbitol, lactose, and surfactants that are acceptable as medical additives.

The aforementioned additives can be added singly or in combination according to the types of formulas as treatment drugs of this invention. For example, when used as an injection formula, a purified decoy nucleic acid is dissolved in a solvent (e.g., saline, buffer solution, glucose solution, etc.) and then mixed with Tween 80, Tween 20, gelatin, and human serum albumin, etc. Alternatively, it can be freeze-dried so that it can be dissolved before use. As a freeze-dry forming agent, the following materials are available: sugars such as mannitol, glucose and lactose, etc., corn, wheat and other vegetable starch, celluloses such as methyl cellulose, hydroxypropylmethyl cellulose, or sodium carboxymethylcellulose, rubbers such as gum Arabic, traganto rubber, gelatin, collagen, etc. If desirable, disintegrants or solubilizers such as cross-linked polyvinyl pyrrolidone, agar-agar, alginic acid or its salts (e.g., sodium alginate).

Doses of the pharmaceutical composition of the present invention vary with age, gender, symptoms, administration routes, frequency of administration, and types of formulas. A method of administration is appropriately selected based on patients' ages and symptoms. An effective dose is the amount of siRNA or shRNA, decoy nucleic acid or antisense oligonucleotide that is required for reducing symptoms of the diseases and conditions. For example, in the case of decoy nucleic acid, the therapeutic effect and toxicity are determined through standard pharmacological procedures using cell cultures and experimental animals: for example, ED₅₀ (therapeutically effective dose at 50% of the population) or LD₅₀ (lethal dose at 50% of the population).

A dose ratio between the therapeutic effect and the toxicity effect is therapeutically efficient and is expressed as ED₅₀/LD₅₀. A single dosage of the pharmaceutical composition of the invention ranges from 0.1 μg to 100 mg per kg bodyweight and preferably from 1 to 10 μg. However, the aforementioned treatment agent is not limited by these dosages. For example, if adeno virus is administered, a single daily dose is approximately 10⁶ to 10¹³ and it is administered with an interval of 1 week to 8 weeks. However, the pharmaceutical compositions of the invention are not limited by these dosages.

This invention will be described in detail with reference to the examples. However, this invention will not be limited by these examples.

### EXAMPLE 1

Study of Induction of Differentiation of Nerve Cells Using siRNA

<Materials and Methods>

Mouse neural blastoma-derived Neuro2a cell line was used in this embodiment. In this case, Neuro2a cells extend axons and differentiate upon removal of serum and addition of a drug. Neuro2a cells were suspended in a Dulbecco's Modified Eagle's Medium (DMEM) supplemented with 10% fetal calf serum (FCS), 1% penicillin/streptomycin and cultured at 37° C. in a 5% CO₂ incubator. Neuro2a cells were seeded (4×10⁴ cells/60 mm dish) and incubated for 24 hours. Two kinds of short interfering RNAs (siRNA) against genes (SEQ ID No. 1 and 2) coding Synoviolin and siRNA against Green Fluorescent Protein (GFP) gene as a control were transfected using Oligofectamine™ Reagent (Invitrogen Corp.).

The final concentration of siRNA was 100 nM and the amount of Oligofectamine™ Reagent used was 8 μL. During the transfection period, the culture medium contained no serum and no antibiotics. After 4 hours from the transfection, incubation was continued with the addition of 10% FCS.

The target sequences for the preparation of siRNA were as follows.

Synoviolin siRNA1:

(SEQ ID No.3)

CGT TCC TGG TAC GCC GTC A
Synoviolin siRNA2:

(SEQ ID No.4)

GAA ATG GTG ACT GGT GCT A
GFP siRNA:

(SEQ ID No.5)

GGC TAC GTC CAG GAG CGC A

<Results>

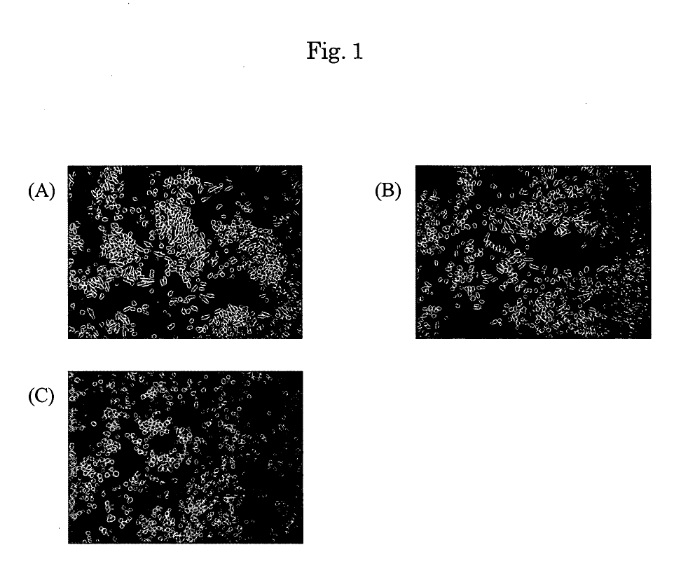

Morphological changes were found 2 days after the transfection in the cells including extension of axons in the cells wherein both siRNA against Synoviolin were transfected (FIG. 1). Compared to the control sample transfected by siRNA against GFP (GFP siRNA) (panel (C) in FIG. 1), the cells transfected by Synoviolin siRNA1 and Synoviolin siRNA2 (respective panel (A) and panel (B) in FIG. 1) exhibited greater numbers of cells with extended axons. Such changes were not found with siRNA against GFP.

### INDUSTRIAL APPLICABILITY

This invention provides a nerve cell differentiation inducing drug containing a Synoviolin expression inhibitor. The present invention's inducing drug is useful for treatments of neural disorders including Alzheimer's disease, Parkinson's disease, peripheral nerve disorders and spinal injury.

Sequence Listing Free Text

SEQ ID No. 5: Synthesized DNA

SEQ ID No. 6:Synthesized DNA

SEQ ID No. 7: Synthesized DNA

1. A nerve cell differentiation induction drug containing a Synoviolin expression inhibitor.

2. The inducing drug as claimed in claim 1 wherein the Synoviolin expression inhibitor is siRNA or shRNA against genes coding Synoviolin.

3. The inducing drug as claimed in claim 2 wherein the gene coding Synoviolin contains a base sequence shown in SEQ ID No. 1 or No. 2.

4. The inducing drug as claimed in claim 2 wherein siRNA targets a portion of the sequence in the base SEQ ID No. 1 and No. 2.

5. The inducing drug as claimed in claim 4 wherein a portion of the sequence has a base sequence shown in SEQ ID No. 3 or No. 4.

6. The inducing drug as claimed in claim 1 wherein the Synoviolin expression inhibitor is a decoy nucleic acid that inhibits the promoter activity by binding to transcription factor of the promoter of the Synoviolin gene.

7. The inducing drug as claimed in claim 6 wherein the decoy nucleic acid is a decoy nucleic acid shown below in (a) or (b).
(a) Decoy nucleic acids consisting of a base sequence indicated by SEQ ID No. 6 or No. 7.
(b) Decoy nucleic acids consisting of a base sequence after deleting 1 or several bases, substituting or adding base sequences in the base sequence indicated by SEQ ID No. 6 or No. 7 and having a function of inhibiting Synoviolin gene promoter activity.

8. The inducing drug as claimed in claim 6 wherein the decoy nucleic acid is a decoy nucleic acid shown below in (a) or (b).
(a) Decoy nucleic acids consisting of a base sequence indicated by SEQ ID No. 6 and No. 7.
(b) Decoy nucleic acids consisting of a base sequence after deleting 1 or several bases, substituting or adding base sequences in the base sequence indicated by SEQ ID No. 6 and No. 7 and having a function of inhibiting Synoviolin gene promoter activity.

9. The inducing drug as claimed in claim 1 wherein the Synoviolin expression inhibitor is an antisense oligonucleotide against the gene coding Synoviolin.

10. The inducing drug as claimed in claim 9 wherein the gene coding Synoviolin contains a base sequence shown in SEQ ID No. 1 or No. 2.

11. The inducing drug as claimed in claim 9 wherein the antisense oligonucleotide targets a portion of the sequence in the base SEQ ID No. 1 and No. 2.

12. (canceled)

13. (canceled)

14. A method of inducing differentiation of nerve cells, comprising inhibiting the expression of Synoviolin.

15. A method for treating a neural disorder, comprising administering the inducing drug according to any one of claims 1 through 11 to a subject in need thereof.

16. The method of claim 15, wherein said neural disorder is selected from the group consisting of Alzheimer's disease, Parkinson's disease, peripheral nerve disorders and spinal injury.
